(19) United States
(12) Patent Application Publication
(10) Pub. No.: US 2022/0155770 A1
(43) Pub. Date: May 19, 2022

(54) Method of Fault Monitoring of Sewage Treatment Process Based on OICA and RNN Fusion Model

(71) Applicant: BEIJING UNIVERSITY OF TECHNOLOGY, Beijing (CN)

(72) Inventors: Peng CHANG, BEIJING (CN); Zeyu LI, BEIJING (CN); Kai WANG, BEIJING (CN); Chunhao DING, BEIJING (CN); Chen JIN, BEIJING (CN); Xiangyu ZHANG, BEIJING (CN); Ruiwei LU, BEIJING (CN); Pu WANG, BEIJING (CN)

(21) Appl. No.: 17/520,378

(22) Filed: Nov. 5, 2021

Related U.S. Application Data
(63) Continuation of application No. PCT/CN2019/125888, filed on Dec. 17, 2019.

(30) Foreign Application Priority Data
Dec. 14, 2019 (CN) 201911298706.X

(51) Int. Cl. G05B 23/02; G05B 13/04; G05B 13/02

(52) U.S. Cl. CPC G05B 23/0254; G05B 23/024; G05B 13/027; G05B 13/04; G05B 23/0281

(57) ABSTRACT

The invent relates to an intelligent fault monitoring method based on high-order information enhanced recurrent neural network, for real-time fault monitoring of sewage treatment process. The invent includes two phases of offline modeling and online monitoring. In offline phase, the original data is extracted into high-dimensional high-order information features using OCIA, which can effectively deal with the non Gaussian feature of the data and solve the correlation between variables. Then the extracted features are trained by DRNN. In the online phase, the data are directly mapped to new high-order feature components, and to be discriminated in category by the DRNN network after trained offline. If there is no fault, then the results get into the monitoring model composed of simple OICA for unsupervised monitoring. If no fault is detected, it is determined that there is no fault in the process. On the contrary, the process fault is determined, and the fault information will be added to the training data of the network for training, so as to continuously improve the monitoring accuracy of DRNN.

### CROSS-REFERENCE TO RELATED APPLICATIONS

This application is a continuation of International Application No. PCT/CN2019/125888, filed on Dec. 17, 2019, which claims priority to Chinese Patent Application No. 201911298706.X, filed on Dec. 14, 2019. The contents of the above applications are hereby incorporated by reference in their entireties and form a part of this specification.

### FIELD OF THE INVENTION

The invention relates to the field of fault monitoring technology based on deep learning, and more particularly, a fault monitoring technology for complex industrial processes. The method based on deep learning in the invention is specific application in typical complex industrial process-fault monitoring of sewage treatment process.

### BACKGROUND TECHNOLOGY

Sewage treatment process is a complex dynamic biochemical process with strong external interference, strong time-varying, strong coupling and nonlinear, so the reliability and stability of the control system is particularly important. But for many abnormal changes (faults) in the process, the controller is often powerless. Due to the continuity and irreplaceable of the sewage treatment system, once the fault occurs, it will cause serious impacts. Due to the characteristics of complex mechanisms and serious environmental interference in sewage treatment process, the data of sewage treatment process has obvious characteristics of nonlinearity, non Gaussian and time correlation. The traditional methods are not effective in fault monitoring of sewage treatment process.

In recent years, the data-driven methods have developed widely. The methods based on data-driven do not need to study the complex mechanism knowledge of sewage treatment process, monitoring results can be obtained in real-time only through the change of process variables, and have been widely used. In the traditional data-driven methods, the multivariate statistical methods are main methods, such as KPCA (kernel principal component analysis) and KPLS (kernel partial least squares, KPLS) and so on. These methods can extract the potential characteristic variables of the process, so as to capture the information of process change, and reflect the occurrence of faults. The methods based on KPCA, KPLS and so on can effectively deal with the non-linearity of data, but the above methods need to assume that the process data obey Gaussian distribution. Due to the interference of complex environment, most data in actual industrial processes do not obey the Gaussian distribution, so these methods are widely limited in practical application. In order to deal with the non Gaussian problem of data, independent component analysis (ICA) is proposed and widely used in the extraction of non Gaussian feature of data. ICA can effectively extract data features using its non Gaussian features. However, ICA requires a large number of iterations in the process of solving and the obtained solution has a high degree of uncertainty, which makes it difficult to apply ICA. At present, there is a lack of effective data processing method to monitor sewage treatment process. In recent years, neural network methods have been widely used in the process monitoring of sewage, such as BP neural network, RBF neural network and so on. Compared with multivariate statistical methods, neural network has stronger nonlinear processing ability, but non Gaussian feature and time correlation of data in the application of sewage monitoring process have not been considered. Moreover, the neural network method is supervised monitoring, and the data label will have certain restrictions on the monitoring of sewage treatment process.

### SUMMARY OF THE INVENTION

In order to overcome the shortcomings of the two technical elements mentioned above. An intelligent fault monitoring method based on high-order information enhanced recurrent neural network is established. In the feature extraction stage, OICA (Overcomplete Independent Component Analysis) is used to extract the original data into high-order information features. The algorithm of OICA is proposed by Anastasia et al in Massachusetts Institute of technology. The algorithm does not need to assume that the data obey Gaussian distribution, and it has low computational complexity and is not restricted by the form of mixed matrix. After that, the feature data extracted by OICA is put into multi-layer recurrent neural network DRNN (Deep Recurrent Neural Network) to be trained layer by layer. Recurrent neural network can learn time series information with multiple abstract levels in data, which is more sensitive to the change of data characteristics and easier to detect fault. While monitoring is operated through DRNN, the extracted high-order statistical information directly establishes a monitoring model for monitoring. Monitoring established directly by OICA is unsupervised monitoring method, and its purpose is to monitor the fault types that are not in the existing label information, and expand the database of existing fault data on the basis of improving the monitoring accuracy rate, so as to gradually improve monitoring results with the increase of the monitoring ability over time.

The technical scheme and implementation steps in the invention are as follows:

### A. Offline Modeling Phase

1) Collect the historical data of the sewage treatment process, and the historical data X is composed of the normal data of the sewage treatment process obtained from the offline test. The data include N sampling times, and J process variables at each sampling time are collected to form a data matrix X=[x₁, x₂, . . . x_{N}]^{T}∈R^{N×J}. Therein, for each sampling time xᵢ=(x_{i,1}, x_{i,2}, . . . , x_{i,j}), x_{i,j} represents the measured value of the jth variable at the ith sampling time;
2) Then, the historical data X is standardized, therein the standardized formula of the jth variable at the ith sampling time is as follows:

$$\overline{{x}_{i , j}} = \frac{{x}_{i , j} - Mean ( j )}{S t d ( j )}$$

Therein, i=1, 2, . . . N, j=1, 2, . . . J; the standardized data in step 2 is reconstructed into a two-dimensional matrix, as shown in the following formula:

$$\overline{X} = [ \begin{matrix}\overline{{x}_{1 , 1}} & … & \overline{{x}_{1 , j}} \\ ⋮ & ⋱ & ⋮ \\ \overline{{x}_{i , 1}} & … & \overline{{x}_{i , j}}\end{matrix} ]$$

3) Using the algorithm of OICA mentioned above, X̅ is mapped to a high-order feature matrix S. The mapped higher-order feature can effectively reflect the non Gaussian feature of the data and provide more fault information. The specific steps are as follows: the unmixing matrix W is calculated by OICA, and then the original data X̅ is mapped into a high-order characteristic matrix S using W. The formula of higher-order characteristic matrix S of X̅ is obtained by W as follows:

S=W^{T}X̅^{T}
Furthermore, the residual E is obtained based on S, and the formula of solving residual is as follows:

E=X̅−WS
4) The statistic I² of independent component space and the statistic SPE of residual space are calculated based on S and E respectively, as follows:

I²=S^{T}S

SPE=E^{T}E
The kernel density estimation algorithm is used to obtain the estimated value Iₗᵢₘᵢₜ² and SPEₗᵢₘᵢₜ of statistics I² and SPE at the preset confidence limit, and take it as the control limit of the subsequent fault monitoring using OICA.
5) Then set up label Y for the historical data X. According to the fault type corresponding to X at each time, normal sewage treatment process is set as 1, while fault process is set as 0.
6) The high-order characteristic matrix S obtained from step 3 and label data Y obtained from step 5 are put into deep recurrent neural network DRNN for supervised training. The input of deep recurrent neural networks is the high-order feature information S obtained by OICA, and the corresponding label data of network input is the fault classification label Y obtained from step 5. the parameters and structure of neurons after supervised training in DRNN are saved.
   B. Online monitoring stage:
1) The preprocessing method of new data during online monitoring is shown in offline step 2, and the processed new data X_{new} is obtained.
2) New high-order feature data S_{new} is obtained from new data X_{new} through the off-line unmixing matrix W

S_{new}=W^{T}X̅_{new}^{T}
3) Put S_{new} as network input into deep recurrent neural network (DRNN) of the trained network parameters in the offline stage to execute operation. An output y will be got through the operation of DRNN neurons of the data, and y is the index data for us to judge there is a fault or not. When y is greater than 0.5, it indicates there is a fault; when y is less than 0.5, it indicates that there is no faults at the present time.
4) The faults can be well supervised classified based on DRNN, but the monitoring performance of the above methods may decrease when there is a fault that does not exists in the training database of DRNN. Furthermore, the algorithm in the invention proposes an unsupervised algorithm based on OICA to monitor the above faults, so as to calibrate the monitoring results obtained by DRNN. When the monitoring results obtained by DRNN are normal, the secondary monitoring is carried out. The specific steps are as follows: firstly, residual E_{new} of the new data X_{new} is obtained through the high-order statistical information S_{new}, as shown in the following formula:

E_{new}=X̅_{new}−WS_{new}
Therein, W is the unmixing matrix determined in step 4);
5) The monitoring statistics Iₖ² and SPEₖ of current sampling time k are calculated, as shown in the following formula:

Iₖ²=S_{new}′S_{new}

SPEₖ=E_{new}′E_{new}
6) The monitoring statistics Iₖ² and SPEₖ obtained from the above steps are compared with the control limit Iₗᵢₘᵢₜ² and SPEₗᵢₘᵢₜ obtained from step 6), if any of the above two indicators exceeds the limit, it is considered that there is a fault and an alarm is given; otherwise, it is considered as normal;
7) The fault data is set up fault label according to offline step 5 and is added into the training database of DRNN for training. The continuous iterative training keeps DRNN learning new fault information all the time.

### Beneficial Effect

Compared with the existing technology, the intelligent fault monitoring method based on the high-order information enhanced recurrent neural network can deal with the non Gaussian feature of the data, and improve the ability of feature extraction for the original data, and its fusion with the recurrent neural network structure can extract the time sequence information of different levels of sewage data, and effectively improve the monitoring accuracy in sewage monitoring. Through the simultaneous monitoring and calibration of OICA unsupervised model, the supervised training data of the fault can be continuously improved, and so does the monitoring accuracy of the whole monitoring model.

### DESCRIPTION OF DRAWINGS

FIG. 1 is the overall flow chart of the algorithm in the invention;

FIG. 2 shows the monitoring chart of bulking fault of sewage and sludge in sunny day;

FIG. 3 shows the monitoring chart of the toxicity impact fault of sewage in sunny day;

FIG. 4 shows the monitoring chart of bulking fault of sewage and sludge in rainy day;

FIG. 5 shows the monitoring chart of the toxicity impact fault of sewage in rainy day;

FIG. 6 is the logic chart of hardware system this method relies on;

FIG. 7 is the schematic chart of the network structure proposed in the method of the present invention.

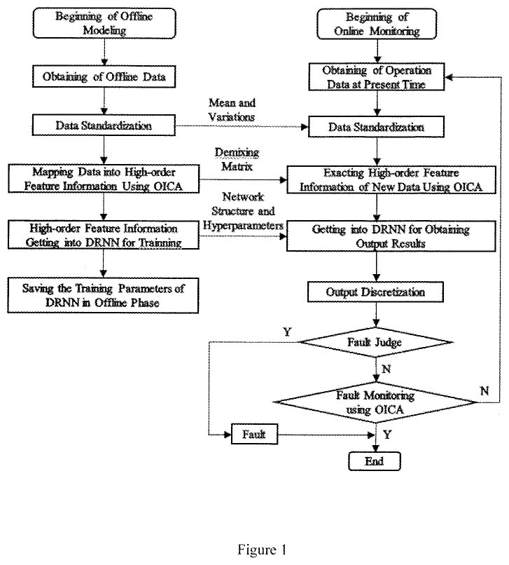

### EXEMPLARY EMBODIMENT

In order to solve the above problems, a method of fault monitoring of sewage treatment process based on OICA and RNN fusion model is proposed, which is based on an online monitoring equipment. The whole equipment includes input module, information processing module, console module and output visualization module. The proposed method is imported into the information processing module, and then the network monitoring model is established using the process data retained in the actual industry, and the established model is saved for online fault monitoring. In the actual online monitoring of industrial process, firstly the real-time process variables collected by the factory data sensors are connected to the input module as the input information of the monitoring equipment, and then the trained model is selected through the console to monitor, and the monitoring results are displayed in real time through the visualization module, so that the on-site staff can take measures in time according to the visual monitoring results, reducing the economic loss caused by process faults.

The process of sewage treatment is extremely complex, including not only all kinds of physical and chemical reactions, but also biochemical reactions. In addition, various uncertain factors, such as influent flow rate, water quality and load changes, etc., have brought great challenges to the establishment of sewage treatment monitoring model. The invention uses “Benchmark Simulation Model 1” developed by IWA as the actual sewage treatment process for real-time simulation. The model consists of five reaction tanks (5999 m³) and one secondary sedimentation tank (6000 m³). In addition, there are three aeration tanks. The aeration tank has 10 layers, 4 meters deep and covers an area of 1500 m². The reaction process includes internal backflow and external backflow. The average sewage treatment flow rate is 20,000 m³/d and the COD is 300 mg/L. The effluent quality index of the sewage model is shown in Table 1. The fault setting model in the invention simulates two kinds of faults based on BSM1 model, sludge bulking fault and toxic impact fault.

TABLE 1
The effluent quality index of the sewage

| Variable | Unit |
|---|---|
| Effluent flow rate | m⁻³ · d |
| The concentration of SI in the Effluent | g COD · m⁻³ |
| The concentration of SS in the Effluent | g COD · m⁻³ |
| The concentration of XI in the Effluent | g COD · m⁻³ |
| The concentration of XS in the Effluent | g COD · m⁻³ |
| The concentration of XBH in the Effluent | g COD · m⁻³ |
| The concentration of XBA in the Effluent | g COD · m⁻³ |
| The concentration of XP in the Effluent | g COD · m⁻³ |
| The concentration of SO in the Effluent | g (−COD) · m⁻³ |
| The concentration of SNO in the Effluent | g N · m⁻³ |
| The concentration of SNH in the Effluent | g N · m⁻³ |
| The concentration of SND in the Effluent | g N · m⁻³ |
| The concentration of XND in the Effluent | g N · m⁻³ |
| The concentration of SALK in the Effluent | mol HCO3− · m⁻³ |
| The concentration of TSS in the Effluent | g SS · m⁻³ |
| The concentration of Kjeldahl N in the Effluent | g N · m⁻³ |

The application process of the invention in the BSM1 simulation platform is described as follows:

### A. Offline Modeling Stage:

Step 1: The invention simulates the sludge bulking fault and toxicity impact fault in the sewage treatment process to verify the algorithm. 14-day data of normal weather and rainstorm are collected by BSM1 model with a sampling interval of 15 minutes and a total of 1344 sampling points for each weather. In the experiment, several batches of sludge bulking data and normal data with different fault degrees under the same type were used for offline training, and a group of new single batch of sludge fault data was trained for test. The training and test data of simulated toxicity impact fault were the same as those of sludge bulking fault.

Step 2: The offline data of sewage treatment process in the normal working condition was processed, and it includes N sampling times collected from multiple batches of data and 16 process variables, which form a data matrix X=[x₁, x₂, . . . x_{N}]^{T}∈R^{N×16}. Therein, for each sampling time xᵢ=(x_{i,1}, x_{i,2}, . . . , x_{i,j}), x_{i,j} represents the measured value of the jth variable at the ith sampling time;

Step 3: Then, the historical data X is standardized, therein the standardized formula of the jth variable at the ith sampling time is as follows:

$$\overline{{x}_{i , j}} = \frac{{x}_{i , j} - Mean ( j )}{S t d ( j )}$$

Therein, i=1, 2, . . . N, j=1, 2, . . . J; the standardized data in step 2 is reconstructed into a two-dimensional matrix, as shown in the following formula:

$$\overline{X} = [ \begin{matrix}\overline{{x}_{1 , 1}} & … & \overline{{x}_{1 , j}} \\ ⋮ & ⋱ & ⋮ \\ \overline{{x}_{i , 1}} & … & \overline{{x}_{i , j}}\end{matrix} ]$$

Step 4: Using the OICA algorithm mentioned above, X̅ is mapped to a high-order feature matrix S. The mapped higher-order feature can effectively reflect the non Gaussian feature of the data and provide more fault information. The specific steps are as follows: the unmixing matrix W is calculated by OICA, and then the original data X̅ is mapped into a high-order characteristic matrix S using W. The formula of higher-order characteristic matrix S of X̅ is obtained by W as follows:

S=W^{T}X̅^{T}

Furthermore, the residual E is obtained based on S, and the formula of solving residual is as follows:

E=X̅−WS

Step 5: The statistic I² of independent component space and the statistic SPE of residual space are calculated based on S and E respectively, as follows:

I²=S^{T}S

SPE=E^{T}E

The kernel density estimation algorithm is used to obtain the estimated value Iₗᵢₘᵢₜ² and SPEₗᵢₘᵢₜ of statistics I² and SPE at the preset confidence limit, and take it as the control limit of the subsequent fault monitoring using OICA.

Step 6: Then set up label Y for the historical data X. According to the fault type corresponding to X at each time, normal sewage treatment process is set as 1, while fault process is set as 0.

Step 7: The high-order characteristic matrix S obtained from step 3 and label data Y obtained from step 5 are put into deep recurrent neural network DRNN for supervised training. The input of deep recurrent neural networks is the high-order feature information S obtained by OICA, and the corresponding label data of network input is the fault classification label Y obtained from step 5. The parameters and structure of neurons in DRNN after supervised training in DRNN are saved. The specific neural network structure and its parameters of DRNN are shown in the table below.

TABLE 1
The neural network structure and its hyper-parameters of DRNN

| Hyper-parameters | Parameter Values |
|---|---|
| Iterations | 100 |
| Number of hidden layers | 3 |
| Number of Neurons in Each Layer of Hidden | 30-20-10 |
| Layer | |
| Learning Rate | 0.01 |

### B. Online Monitoring Stage:

Step 8 The preprocessing method of new data during online monitoring is shown in offline step 3, and the processed new data X_{new} is obtained.

Step 9 New high-order feature data S_{new} is obtained from new data X_{new} through the off-line unmixing matrix W

S_{new}=W^{T}X̅_{new}^{T}

Step 10 Put S_{new} as network input into deep recurrent neural network (DRNN) of the trained network parameters in the offline stage to execute operation. An output y will be got through the operation of DRNN neurons of the data, and y is the index data for us to judge there is a fault or not. When y is greater than 0.5, it indicates there is a fault; when y is less than 0.5, it indicates that there is no faults at the present time.

Step 11: The faults can be well supervised classified based on DRNN, but the monitoring performance of the above methods may decrease when there is a fault that does not exists in the training database of DRNN. Furthermore, the algorithm in the invention proposes an unsupervised algorithm based on OICA to monitor the above faults, so as to calibrate the monitoring results obtained by DRNN. When the monitoring results obtained by DRNN are normal, the secondary monitoring is carried out. The specific steps are as follows: firstly, residual E_{new} of the new data X_{new} is obtained through the high-order statistical information S_{new}, as shown in the following formula:

E_{new}=X̅_{new}−WS_{new}

Therein, W is the unmixing matrix determined in step 4);

Step 12: The monitoring statistics Iₖ² and SPEₖ of current sampling time k are calculated, as shown in the following formula:

Iₖ²=S_{new}′S_{new}

SPEₖ=E_{new}′E_{new}

Step 13: The monitoring statistics Iₖ² and SPEₖ obtained from the above steps are compared with the control limit Iₗᵢₘᵢₜ² and SPEₗᵢₘᵢₜ obtained from step 6), if any of the above two indicators exceeds the limit, it is considered that there is a fault and an alarm is given; otherwise, it is considered as normal;

Step 15: The fault data is set up fault label according to offline step 5 and is added into the training database of DRNN for training. The continuous iterative training keeps DRNN learning new fault information all the time.

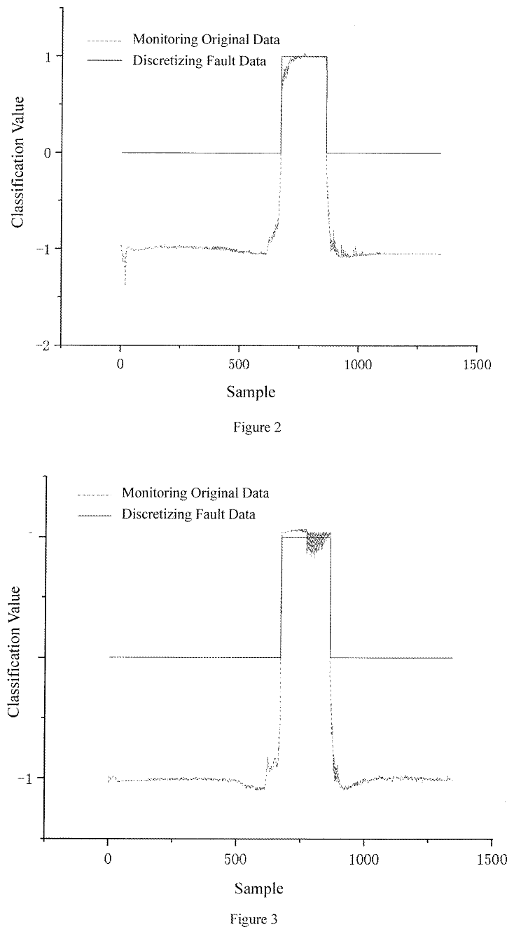

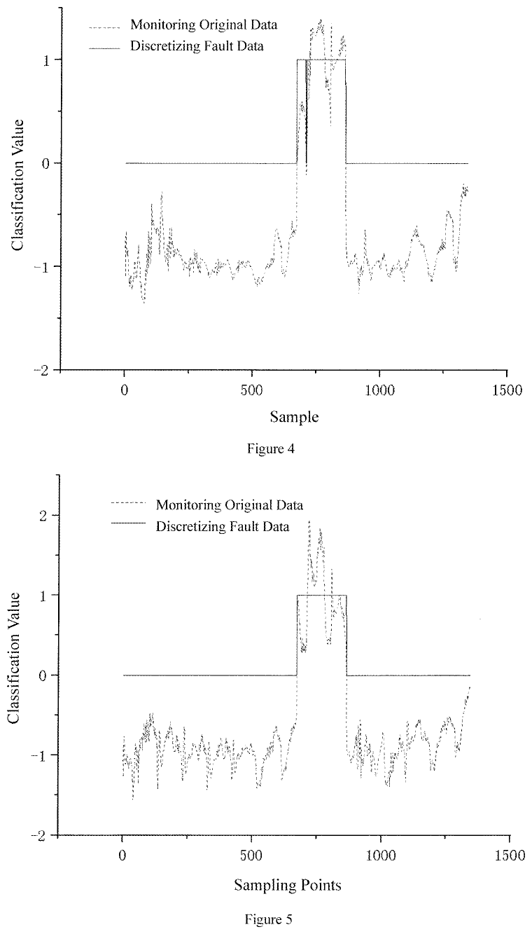

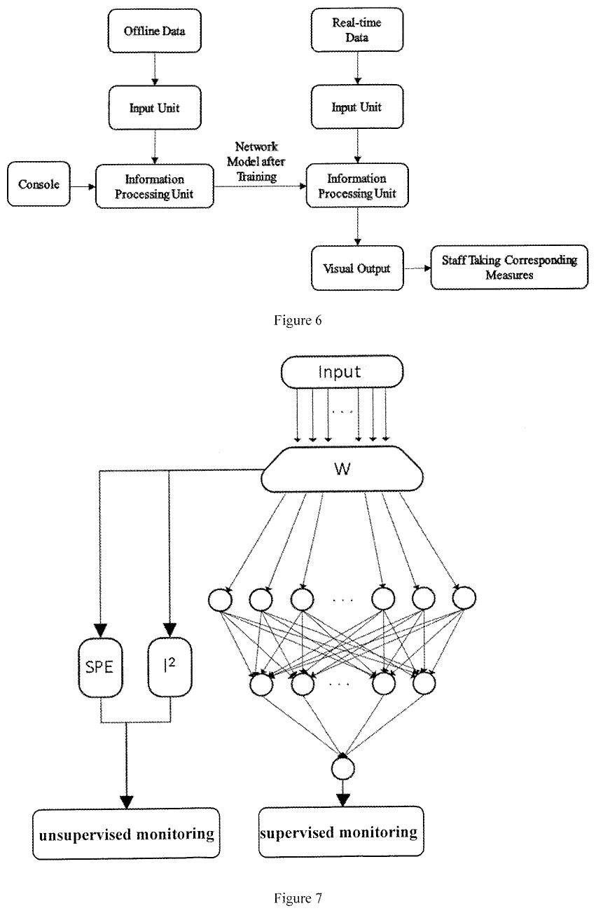

The above are the specific application steps of the fault monitoring of the sewage treatment process on the BSM1 sewage simulation platform. In order to verify the effectiveness of the method, the invention respectively sets up two kinds of faults of sludge bulking and toxicity impact of sewage in sunny days and in rainy days to test the monitoring accuracy of the invention under different weather conditions. FIGS. 2-5 are the monitoring charts of sludge bulking in sunny days and rainy days respectively, and 1 in the discrete classification value in the chart represents the occurrence of fault. Table 1 shows the alarm time, false alarm rate and missed alarm rate of the faults. It can be seen from FIGS. 2-5 and table 1 that the method of the invention can effectively monitor the occurrence of sludge fault, and has low missed alarm rate and false alarm rate. In addition, the method also has good monitoring performance in the complex environment such as rainy days, indicating that the invention has strong robustness.

TABLE 2
The monitoring performance of the invent under different conditions

| | | | | Number of |
|---|---|---|---|---|
| | Fault | Alarm | Number of | Missed |
| Type of Faults | Time | Time | False Alarm | Alarm |
| Bulking Fault of | 672-864 | 672 | 0 | 1 |
| Sludge in Sunny | | | | |
| Days | | | | |
| Toxicity Impact | 672-864 | 672 | 3 | 1 |
| Fault in Sunny Days | | | | |
| Bulking Fault of | 672-864 | 672 | 1 | 2 |
| Sludge in Rainny | | | | |
| Days | | | | |
| Toxicity Impact | 672-864 | 672 | 0 | 1 |
| Fault in Rainny | | | | |
| Days | | | | |

We claim:

1. A method of fault monitoring of sewage treatment process based on OICA and RNN fusion model, comprising an offline modeling phase and an online monitoring phase, the specific steps are as follows:
A. offline modeling stage:
1) collect historical data X of the sewage treatment process, and the historical data X is composed of normal data of the sewage treatment process obtained from offline test, the data include N sampling times, and J process variables at each sampling time are collected to form a data matrix X=[x₁, x₂, . . . x_{N}]^{T}∈ [symbol] ^{N×J}, therein, xᵢ=(x_{i,1}, x_{i,2}, . . . , x_{i,j}), x_{i,j} represents measured value of jth variable at ith sampling time;
2) then, the historical data X is standardized, therein standardized formula of the jth variable at the ith sampling time is as follows:
$$\overline{{x}_{i , j}} = \frac{{x}_{i , j} - Mean ( j )}{S t d ( j )}$$
therein, i=1, 2, . . . N, j=1, 2, . . . J; the standardized data in step 2 is reconstructed into a two-dimensional matrix, as shown in the following formula:
$$\overline{X} = [ \begin{matrix}\overline{{x}_{1 , 1}} & … & \overline{{x}_{1 , j}} \\ ⋮ & ⋱ & ⋮ \\ \overline{{x}_{i , 1}} & … & \overline{{x}_{i , j}}\end{matrix} ]$$
3) X̅ is mapped to a high-order feature matrix S using the algorithm of OICA, and the specific steps are as follows: an unmixing matrix W is calculated by OICA, and then the original data X̅ is mapped into a high-order characteristic matrix S using W, a formula of higher-order characteristic matrix S of X̅ is obtained by W as follows:
S=W^{T}X̅^{T}
furthermore, residual E is obtained based on S, and a formula of solving residual is as follows:
E=X̅−WS
4) statistic I² of independent component space and statistic SPE of residual space are calculated based on S and E respectively, as follows:
I²=S^{T}S
SPE=E^{T}E
a kernel density estimation algorithm is used to obtain estimated value Iₗᵢₘᵢₜ² and SPEₗᵢₘᵢₜ of statistics I² and SPE at a preset confidence limit, and take it as a control limit of subsequent fault monitoring using OICA;
5) then set up label Y for the historical data X, namely normal and fault;
6) the high-order characteristic matrix S obtained from step 3 and label data Y obtained from step 5 are put into deep recurrent neural network DRNN for supervised training; parameters and structure of neurons after supervised training by DRNN are saved;
B. online monitoring stage:
1) a preprocessing method of new data during online monitoring is shown in offline step 2, and processed new data X_{new} is obtained;
2) new high-order feature data S_{new} is obtained from new data X_{new} through the off-line unmixing matrix W
S_{new}=W^{T}X̅_{new}^{T}
3) put S_{new} into a trained deep recurrent neural network (DRNN) in the offline stage to judge there is a fault or not; when the fault index data is greater than 0.5, it indicates there is fault, when the fault index data is less than 0.5, it indicates that it is normal;
4) when monitoring results obtained by DRNN are normal, secondary monitoring is carried out: firstly, residual E_{new} of the data X_{new} is calculated, as shown in the following formula:
E_{new}−X̅_{new}−WS_{new}
therein, W is the unmixing matrix determined in step 4);
5) the monitoring statistics Iₖ² and SPEₖ of current sampling time k are calculated, as shown in the following formula:
Iₖ²=S_{new}′S_{new}
SPEₖ=E_{new}′E_{new}
6) the monitoring statistics Iₖ² and SPEₖ obtained from the above steps are compared with the control limit Iₗᵢₘᵢₜ² and SPEₗᵢₘᵢₜ obtained from step 6) in offline monitoring phase, if any of the above two indicators exceeds the limit, it is considered that there is a fault and an alarm is given; otherwise, it is considered as normal;
7) the fault data is set up fault label according to offline step 5 and is added into the training database of DRNN for training, DRNN is trained again using the updated training data for learning new fault information, so as to monitor accurately.

2. The method of fault monitoring of sewage treatment process based on OICA and RNN fusion model according to claim 1, wherein the loss function of deep recurrent neural network (DRNN) is cross entropy loss function.
